(19) United States
(12) Patent Application Publication
(10) Pub. No.: US 2017/0079284 A1
(43) Pub. Date: Mar. 23, 2017

(54) APPETITE-SUPPRESSING FOOD PRODUCT CONTAINING JERUSALEM ARTICHOKE FLOUR

(71) Applicant: IPC Intellectual Property Capital Deutschland GmbH, Hamburg (DE)

(72) Inventors: Dirk Sinkus, Hamburg (DE); Siedfried Becker, Hamburg (DE); Volker Rodemer, Hamburg (DE)

(73) Assignee: IPC Intellectual Property Capital Deutschland GmbH, Hamburg (DE)

(21) Appl. No.: 15/126,198

(22) PCT Filed: Mar. 16, 2015
(86) PCT No.: PCT/IB2015/001043

§ 371 Date: Sep. 14, 2016

Related U.S. Application Data
(60) Provisional application No. 61/953,576, filed on Mar. 14, 2014.

(51) Int. Cl. A21D 2/02; A23L 27/00; A61K 36/23; A23L 33/22; A61K 31/695; A61K 36/53; A21D 2/36; A23L 33/125

(52) U.S. Cl. CPC A21D 2/02; A21D 2/36; A21D 2/366; A23L 27/88; A23L 33/125; A23L 33/22; A61K 31/695; A61K 36/53; A61K 36/23; A23V 2002/00

(57) ABSTRACT

Food products, such as a dough for producing a baked product, that contain Jerusalem artichoke flour, a flavor enhancer, and an anti-flatulence agent are disclosed. In certain embodiments, the food product is a baked product that is a cookie or cracker. In accordance with the subject matter of the disclosure, in certain embodiments, the food product contains at least 10 weight percent Jerusalem artichoke flour. The food product also may contain at least 1 weight percent of a flavor enhancer to mask the unpleasant aftertaste of the cynarin that is naturally present in the Jersualem artichoke flour. The food product additionally contains at least one anti-flatulence agent.

### CROSS-REFERENCE TO RELATED APPLICATION

This application claims the benefit of priority of U.S. Provisional Application Ser. No. 61/953,576 filed Mar. 14, 2014, which is incorporated herein by reference in its entirety.

### BACKGROUND OF THE INVENTION

Jersusalem artichoke flour is a natural appetite suppressant due, in part, to the presence of inulin. Jerusalem artichoke flour has been successfully incorporated into baked products, such as cookies, in flour form. Despite its appetite-suppressing effect, Jerusalem artichoke flour contains cynarin, which leaves an unpleasant aftertaste. Moreover, incorporation of the Jerusalem artichoke flour into the flour of baked goods has been known to cause flatulence and other discomfort.

### SUMMARY OF THE INVENTION

A food product containing Jerusalem artichoke flour that has a favorable taste and reduced flatulence effects is needed. Embodiments of the disclosed subject matter relate to food products, such as a dough for producing a baked product, that contain Jerusalem artichoke flour, fat, a sweetener, and an anti-flatulence agent. In certain embodiments, the food product is a dough that contains at least 10 weight percent Jersualem artichoke flour and an anti-flatulence agent. One embodiment of the disclosed subject matter is a baked product obtained from baking this dough. In a particular embodiment, this can be a cookie or cracker.

In certain embodiments, the instant application relates to a composition, wherein the composition is a food product comprising Jerusalem artichoke flour, a flavor enhancer, and an effective amount of an anti-flatulence agent. In some embodiments, the food product comprises about 4 to 20 weight percent Jerusalem artichoke flour. In certain embodiments, the food product further comprises a second flour selected from the group consisting of: wheat flour, rye flour, and spelt flour. In a particular embodiment, the second flour is wheat flour. In further embodiments, the ratio of Jerusalem artichoke flour to wheat flour is about 30%. In some embodiments, the proportion of total flour in the food product is about 30 to 60 weight percent.

In other embodiments, the composition of the instant invention further comprises a fat component. In certain embodiments, the fat component comprises about 10 to 30 weight percent of the composition.

In some embodiments, the inventive composition further comprises an additive selected from the group consisting of: eggs, egg by-products, milk, and milk by-products.

In certain embodiments, the flavor enhancer of the composition is a sweetener selected from the group consisting of: fruit syrups, concentrated fruit juices, fruit juices, hazelnut syrup, honey, sugars, and other high sugar content products. In other embodiments, the flavor enhancer is a salt. In yet other embodiments, the flavor enhancer is a spice. In some embodiments, the flavor enhancer is selected from the group consisting of cocoa and vanilla. In certain embodiments, the vanilla is selected from the group consisting of: vanilla extract, artificial vanillin, vanilla sugar, and combinations thereof. In other embodiments, the flavor enhancer is selected from nuts, raisins, coconut, chocolate chips, butterscotch chips, and caramel chips.

In certain embodiments, the anti-flatulence agent of the composition is selected from synthetic and organic anti-flatulence agents. In some embodiments, the anti-flatuence agent is dimethicone or simethicone. In a particular embodiment, the anti-flatulence agent is dimethicone. In further embodiments, the composition comprises dimethicone in amounts between about 0.1 to 10 ppm. In other embodiments, the anti-flatulence agent is simethicone. In another particular embodiment, the composition comprises about 0.00005 to 0.005 weight percent simethicone. In some embodiments, the organic anti-flatulence agent is selected from the group consisting of: rosemary extract, caraway seeds, fennel, anise, cumin, coriander, thyme, and parsley.

In certain embodiments, the composition is a food product that is a dough, wherein the dough is in a baked form. In further embodiments, the baked form is a cookie or cracker. In some embodiments, the cookie is selected from the group consisting of: chocolate cookies, vanilla cookies, milk cookies, butter cookies, biscuits, chocolate chip cookies, oatmeal cookies, fruit cookies, sugar cookies, animal crackers, and sandwich cookies.

In yet other embodiments, the anti-flatulence agent is added to a composition of the invention, wherein the composition is a dough and the anti-flatulence agent is added to the dough prior to baking. In other embodiments, the anti-flatulence agent is added to the dough after it has been baked. In further embodiments, the anti-flatulence agent is sprayed onto the baked product. In particular embodiments, the sprayed-on anti-flatulence agent is selected from the group consisting of: dimethicone and simethicone.

### DETAILED DESCRIPTION

Embodiments of the disclosed subject matter include a dough for producing a baked product that contains Jerusalem artichoke flour, fat, a sweetener, and an anti-flatulence agent.

Food products produced in accordance with the present invention exhibit an appetite-suppressing effect, pleasant taste, and reduced flatulence. In particular embodiments, the food product is a baked product. A baked product is a consumable food product that has been baked, particularly a pastry such as cookies or the like. The term “flour” describes any type of flour standard in the production of food products, such as wheat flour, rye flour, or spelt flour, including the Jerusalem artichoke flour added according to this invention.

The Jerusalem artichoke flour employed in the present invention contains, in addition to other constituents, inulin. Inulin is a β-2-fructofuranose material and has been used as a food supplement. Inulin is a prebiotic. That is, inulin is a food material that is metabolized in the intestine by bacteria, e.g., Bifidobacteria and Lactobacilli.

Inulin cannot be broken down in the gastrointestinal tract by most people. However, certain intestinal flora contain enzymes that can break down inulin. This de-composition process oftentimes causes flatulence.

This flatulence problem is solved with the food products of the instant invention, such as baked products produced by a dough of the present invention. To produce a baked product of the instant invention, in certain embodiments, the dough contains a flour portion containing at least 10 weight percent Jerusalem artichoke flour, at least 1 weight percent of a flavor enhancer to mask the taste of the cynarin present in the Jersusalem artichoke flour, and an anti-flatulence agent.

The Jerusalem artichoke contains, among other constituents, inulin, which is known by the state of the art as an appetite-suppressing substance. With the Jerusalem artichoke flour, inulin as a natural raw material can be introduced into the baked product.

The total mass of the dough or other food product can contain Jerusalem artichoke flour in any suitable amount. Typically, Jerusalem artichoke flour will be present in a food product of the instant invention in an amount sufficient to suppress appetite in an individual that consumes the food product. In addition, while the compositions of the instant invention typically contain Jerusalem artichoke (Helianthus tuberosus) in the form of a flour, in some embodiments, other forms of Jerusalem artichoke (e.g., liqueur, juice, whole tuber) may be used either alone or in combination with Jerusalem artichoke flour in a food product of the instant invention to achieve an appetite suppression effect of the food product.

In some embodiments, an appetite suppression effect is achieved in an individual after consumption of two or more food products of the application. For example, in some embodiments, an appetite-suppressing serving size of a food product of the instant invention, such as a cookie, is two helpings of the food product (e.g., two cookies).

In some embodiments, the Jerusalem artichoke flour is present in a dough or other food product in an amount of about 4 to 20 weight percent. In certain embodiments, the Jerusalem artichoke flour can be present in an amount of about 5 to 15 weight percent. In some embodiments, the Jersualem artichoke flour can be present in an amount of about 6 to 12 weight percent.

The proportion of flour in the total mass of the dough or other food product in accordance with the invention can comprise any suitable amount for preparing the dough or other food product for consumption, such as, for example, about 30 to 70 weight percent. In certain embodiments, the proportion of flour in the total mass of the dough or other food product is 40 to 50 weight percent.

The food products of the instant application comprise an anti-flatulence agent. The anti-flatulence agent should be present in the compositions of the instant invention in amounts effective to reduce or prevent flatulence in a subject due to the presence of inulin in the Jerusalem artichoke flour of the inventive doughs and other food products of the instant application. The anti-flatulence agent can be one or more of any suitable compound effective in reducing or preventing flatulence in a subject. In certain embodiments, the anti-flatulence agent is a synthetic compound, such as dimethicone (polydimethylsiloxane) or simethicone (a mixture of polydimethylsiloxane and silica gel). In other embodiments, the anti-flatulence agent is a naturally-occurring composition, such as an herb containing one or more bitter substances, including fennel, anise, caraway, cumin, coriander, thyme, and parsley. In one embodiment, the anti-flatulence agent is rosemary or rosemary extract.

In certain embodiments, a food product of the invention, such as a dough, contains a combination of two or more anti-flatulence agents in amounts that collectively are effective to reduce or prevent flatulence in a subject due to the presence of inulin in the Jerusalem artichoke flour of the inventive compositions of the instant application. For example, in certain embodiments, a food product of the invention contains a combination of two or more synthetic and/or naturally-occurring anti-flatulence compounds. In some embodiments, a food product, such as a baked product such as a cookie or cracker, contains a combination of two or more anti-flatulence agents selected from dimethicone, simethicone, rosemary, and caraway seeds.

The amount of anti-flatulence agent in an inventive dough or other food product of the application can be any suitable amount effective to reduce or prevent flatulence in a subject that consumes the food product, such as with respect to flatulence associated with inulin content in a dough or other food product of the invention. In certain embodiments, the food product contains the anti-flatulence agent in an amount of about 0.005 to 5 weight percent of the food product. In some embodiments, the anti-flatulence agent is present in an amount of about 1 to 3 weight percent of the food product.

Where dimethicone is used, suitable amounts include dimethicone added to the dough or other food product in amounts between about 0.1 to 10 ppm. In certain embodiments, dimethicone is added in trace amounts of less than 0.1 ppm. In embodiments where simethicone is added to the dough or other food product, simethicone can be present in amounts between about 0.00005 to 0.005 weight percent.

The anti-flatulence agent of the compositions of the instant invention may be added to the composition as it is being prepared for consumption, for example, mixed into a dough prior to baking, or after the food product has already been prepared, for example, added to a dough after it has been fully baked, such as by spraying the anti-flatulence agent onto the outside of the fully baked product (e.g., spraying a baked good with dimethicone after the baking process is complete).

The dough or other food product may also contain a sweetener. The sweetener can be selected from sugars and other products with high sugar content, such as honey.

The dough or other food product may also contain a fat component. The fat component can be of plant or animal origin, such as vegetable fats, margarine, butter, and butter fats. Vegetable fats are advantageous due to their quick baking qualities. The proportion of the fat in the total mass of the dough or other food product can be 10 to 40 weight percent. In certain embodiments, the proportion of fat is 15 to 30 weight percent of the dough or other food product. In particular embodiments, the proportion of fat is 20 to 25 weight percent of the dough or other food product.

The food product typically also contains a flavor enhancer. In certain embodiments, the food product comprises a flavor enhancer at 1 weight percent to mask the cynarin taste from the Jerusalem artichoke flour. Any flavor enhancer or combination of flavor enhancers that is effective in masking the cynarin taste from the Jerusalem artichoke flour may be employed in the food products of the instant invention. The flavor enhancer may be naturally derived (e.g., organic) or synthetic.

In some embodiments, the flavor enhancer can be a salt, herb, or spice. In other embodiments, the flavor enhancer can be a sweetener. The flavor enhancer can be selected from fruit syrups, concentrated fruit juices, fruit juices, and hazelnut syrup. Other examples of suitable flavor enhancers include cocoa, vanilla extract, and other natural flavorings. Additional examples of suitable flavor enhancers include artificial flavorings such as synthetic vanillin and other artificial flavors. Further examples of flavor enhancers include nuts, raisins, coconut, chocolate chips, butterscotch chips, and caramel chips. Flavor enhancers such as these can compensate, in certain embodiments, for the undesirable taste generated by the cynarin that is contained in the Jerusalem artichoke flour.

In some embodiments, the flavor enhancer serves both as an anti-flatulence agent and a flavor enhancer (e.g., rosemary, caraway seeds) and is present in a food product of the instant invention in amounts effective to both reduce or prevent flatulence and to mask the cynarin aftertaste from the Jerusalem artichoke flour. In some embodiments, the effective amount of the flavor enhancer is obtained in combination with one or more different flavor enhancers.

The proportion of the flavor enhancer in the total mass of the food product can be any suitable amount effective in masking the cynarin taste from the Jerusalem artichoke flour. In certain embodiments, the flavor enhancer is present in the food product in an amount of about 1 to 6 weight percent. In certain embodiments, the flavor enhancer is present in the food product in an amount of about 2 to 5 weight percent. In some embodiments, the flavor enhancer is present in amounts of less than 1 weight percent, for example, about 0.001 to 0.9 weight percent.

The food products, such as baked products, of the invention can also contain other additives typical of food products. Examples of suitable additives include dried or powdered eggs and milk. Additional examples of suitable additives include milk by-products and egg or egg by-products.

In embodiments where the appetite-suppressing food product is a dough, the dough can be baked into edible products, such as a cookie or cracker. In embodiments where the dough is baked into a cookie, the cookie may also be molded into a variety of shapes. Examples of such shapes include round, square, triangular, elliptical, and the like.

In other embodiments where the appetite-suppressing food product is a dough, the dough may also be used for the production of rotary-molded chocolate cookies, vanilla cookies, milk cookies, butter cookies, biscuits, chocolate chip cookies, oatmeal cookies, fruit cookies, sugar cookies, animal crackers, sandwich cookies, and the like.

The invention will now be further described by way of the following non-limiting examples.

### Example 1

The following ingredients are mixed into dough:

| 300 | g | wheat flour |
|---|---|---|
| 100 | g | topinambour (Jerusalem artichoke) flour |
| 2 | | eggs |
| 200 | g | vegetable fat |
| 5 | g | milk |
| 10 | g | salt |
| 2.5 | mg | simethicone |

Cookies can be produced when baked from this dough.

### Example 2

The following ingredients are mixed into dough:

Sweet:

| 300 | g | wheat flour |
|---|---|---|
| 100 | g | topinambour (Jerusalem artichoke) flour |
| 100 | g | honey |
| 10 | g | vanilla sugar |
| 0.5 | g | salt |
| 2 | | eggs |
| 200 | g | vegetable fat |
| 5 | g | milk |
| 5 | mg | dimethicone (dimethylpolysiloxane) |
| 25 | g | concentrated pear juice |

Cookies can be produced when baked from this dough.

The following ingredients are mixed into dough:

Salty:

| 300 | g | wheat flour |
|---|---|---|
| 100 | g | topinambour (Jerusalem artichoke) flour |
| 2 | | eggs |
| 200 | g | vegetable fat |
| 5 | g | milk |
| 10 | g | salt |
| 5 | mg | dimethicone (dimethylpolysiloxane) |

Cookies can be produced when baked from this dough.

### Example 3

The following ingredients are mixed into dough:

| 300 | g | wheat flour |
|---|---|---|
| 100 | g | topinambour (Jerusalem artichoke) flour |
| 2 | | eggs |
| 200 | g | vegetable fat |
| 5 | g | milk |
| 10 | g | salt |
| 100 | mg | rosemary extract |

Cookies can be produced when baked from this dough.

The following ingredients are mixed into dough:

| 300 | g | wheat flour |
|---|---|---|
| 100 | g | topinambour (Jerusalem artichoke) flour |
| 2 | | eggs |
| 200 | g | vegetable fat |
| 5 | g | milk |
| 10 | g | salt |
| 10 | g | caraway seeds |

Cookies can be produced when baked from this dough.

Although the disclosed subject matter has been described and illustrated with respect to embodiments thereof, it should be understood by those of ordinary skill in the art that features of the disclosed embodiments can be combined, rearranged, etc., to produce additional embodiments within the scope of the invention, and that various other changes, omissions, and additions may be made therein and thereto, without parting from the spirit and scope of the present invention.

Any patent, patent application, or publication cited or described in the present application is hereby incorporated by reference in its entirety as if each individual patent, patent application, or publication was specifically and individually indicated to be incorporated by reference.

What is claimed is:

1. A composition, wherein the composition is a food product comprising:
(a) Jerusalem artichoke flour;
(b) a flavor enhancer; and
(c) an effective amount of an anti-flatulence agent.

2. The composition of claim 1, comprising about 4 to 20 weight percent Jerusalem artichoke flour.

3. The composition of claim 2, further comprising a second flour selected from the group consisting of: wheat flour, rye flour, and spelt flour.

4. The composition of claim 3, wherein the second flour is wheat flour.

5. The composition of claim 4, wherein the ratio of Jerusalem artichoke flour to wheat flour is about 30%.

6. The composition of claim 3, wherein the proportion of total flour in the food product is about 30 to 60 weight percent.

7. The composition of claim 3, further comprising a fat component.

8. The composition of claim 7, wherein the fat component comprises about 10 to 30 weight percent of the composition.

9. The composition of claim 7, further comprising an additive selected from the group consisting of: eggs, egg by-products, milk, and milk by-products.

10. The composition of claim 1, wherein the flavor enhancer is a sweetener selected from the group consisting of: fruit syrups, concentrated fruit juices, fruit juices, hazelnut syrup, honey, sugars, and other high sugar content products.

11. The composition of claim 1, wherein the flavor enhancer is a salt.

12. The composition of claim 1, wherein the flavor enhancer is a spice.

13. The composition of claim 1, wherein the flavor enhancer is selected from the group consisting of cocoa and vanilla.

14. The composition of claim 13, wherein the vanilla is selected from the group consisting of: vanilla extract, artificial vanillin, vanilla sugar, and combinations thereof.

15. The composition of claim 1, wherein the flavor enhancer is selected from the group consisting of: nuts, raisins, coconut, chocolate chips, butterscotch chips, and caramel chips.

16. The composition of claim 1, wherein the anti-flatulence agent is selected from synthetic and organic anti-flatulence agents.

17. The composition of claim 16, wherein the anti-flatuence agent is dimethicone or simethicone.

18. The composition of claim 17, wherein the anti-flatulence agent is dimethicone.

19. The composition of claim 18, wherein the composition comprises dimethicone in amounts between about 0.1 to 10 ppm.

20. The composition of claim 17, wherein the anti-flatulence agent is simethicone.

21. The composition of claim 20, wherein the composition comprises about 0.00005 to 0.005 weight percent simethicone.

22. The composition of claim 16, wherein the organic anti-flatulence agent is selected from the group consisting of: rosemary extract, caraway seeds, fennel, anise, cumin, coriander, thyme, and parsley.

23. The composition of claim 1, wherein the food product is a dough, wherein the dough is in a baked form.

24. The composition of claim 23, wherein the baked form is a cookie or cracker.

25. The composition of claim 24, wherein the cookie is selected from the group consisting of: chocolate cookies, vanilla cookies, milk cookies, butter cookies, biscuits, chocolate chip cookies, oatmeal cookies, fruit cookies, sugar cookies, animal crackers, and sandwich cookies.

26. The composition of claim 23, wherein the anti-flatulence agent is added to the dough prior to baking.

27. The composition of claim 23, wherein the anti-flatulence agent is added to the dough after it has been baked.

28. The composition of claim 27, wherein the anti-flatulence agent is sprayed onto the baked product.

29. The composition of claim 28, wherein the anti-flatulence agent is selected from the group consisting of: dimethicone and simethicone.
